(19) United States
(12) Patent Application Publication
(10) Pub. No.: US 2013/0216484 A1
(43) Pub. Date: Aug. 22, 2013

(54) BIOENZYMATIC TABLET

(71) Applicant: John Ibrahim Khouri, Chicago, IL (US)

(72) Inventor: John Ibrahim Khouri, Chicago, IL (US)

(21) Appl. No.: 13/399,069

(22) Filed: Feb. 17, 2012

(51) Int. Cl. A61K 9/00; A61K 9/28

(52) U.S. Cl. 424/43; 427/2.14

(57) ABSTRACT

It is contemplated that the tablet of the present invention is primarily utilized cleaning of dental instruments. In application a receptacle, such as sonic cleansing machine, is provided with a supply of water. An effervescent tablet containing a bioenzymatic compound is introduced to the water to create a cleaning solution. Soiled dental equipment may be placed in the cleaning solution and the bioenzymatics present in the tablet identify the nature of the waste present on the soiled instruments and begin to produce the enzymes necessary to breakdown the waste thereby cleaning the instruments.

### CROSS-REFERENCE TO RELATED APPLICATION

Not Applicable.

### FEDERALLY SPONSORED RESEARCH OR DEVELOPMENT

Not Applicable

### FIELD OF THE INVENTION

The present invention generally relates to cleaning tablets containing a sufficient amount of bioenzymatics. The tablets are used in creating a cleaning solution for various instruments. That is, the bioenzymatics of the tablet, when dissolved in a solution and presented with a supply of organic material, such waste material deposited on dental instruments in the form of, for example, blood, will self identify the nature of the waste material and thereafter manufacture appropriate enzymes to breakdown and eliminate that waste material. Thus, the tablets may be used to clean most any variety of organic material from instruments even when the identity or concentration of the particular organic material to be cleaned is unknown.

### BACKGROUND OF THE INVENTION

In a typical dental office, various instruments are used to clean and repair teeth. In so doing, the instruments become soiled with a wide variety of organic waste products such as saliva, blood, fats, carbohydrates and the like. Due to cross contamination concerns, all of the instruments must be cleaned and sterilized after use.

Typically, in order to clean and sterilize the instruments, two devices are utilized, an ultrasonic cleaning machine and an autoclave. The cleaning process begins with the ultrasonic cleaning machine. That device utilizes a cleaning solution as well as ultrasonic energy waves to dissolve and remove organic waste material from the instruments. The process begins by filling the ultrasonic machine with a fluid, generally water, and then mixing a cleanser in the fluid to create a cleaning solution. In the past, typical cleansers were provided in liquid or granulated form. However, measuring our precise quantities of cleansers was tedious and inaccurate, often leading to insufficient amounts of cleanser or excessive and wasteful use of the cleanser. As a solution, tablets of cleansers were manufactured. These tablets contained premeasured doses of active ingredients that could easily be added to the ultrasonic cleaning machine. On such tablet is described in U.S. Pat. No. 5,529,788.

Once the cleaning solution was formed, the soiled instruments were added to the machine and ultrasonic energy was introduced to the system for a prescribed period of time. Once complete, the instruments, now generally clean, but not sufficiently sterilized could be placed in specialized packages and then placed into an autoclave. The autoclave could then be used to sterilize the instruments.

Past cleaning tablets, such as the tablet of U.S. Pat. No. 5,529,788 relied on the use of measured concentrations of enzymes as part of the cleaning tablet. For example, a cleaning tablet could contain 45% carrier material, such as sodium bicarbonate and 24% acid, effective for creating an effervescent reaction when dissolved in water, along with 23% granulated protease enzyme, and a balance of surfactant, color dye and fragrance. The drawback of such a tablet is that the total amount of enzyme in a cleaning solution created through the use of such a tablet is determined by the concentration of enzyme present in the tablet. Moreover, the identity of the enzymes present in any cleaning solution is dictated by the enzymes present in the tablet. That is, were the tablet created solely using a concentration of a particular enzyme, a protease enzyme for example, the solution would only contain protease enzymes.

Yet it extremely difficult if not impossible to predetermine the precise concentration and identities of the enzymes needed to clean an unknown quantity of soiled instruments. The amount and identity of waste present on a set of instruments at the conclusion of one particular patient's dental visit varies significantly from the amount of waste present on instruments used with another patient. Using enzymatic cleaning tablets results in the danger that the particular enzymes necessary to breakdown and clean a given number of instruments will either fail to be included in the solution or will not be included in sufficient concentration to adequately clean the instruments.

An additional drawback of prior tablets is that those tablets failed to account for the varying levels of water hardness found in different locals and did not provide any means for preventing the corrosion of the instruments. As a result, persons utilizing the tablets to form cleaning solutions, such as a solution in an ultrasonic cleaning machine, were required to either fill the machine with distilled water, allow for mineral deposits to be present on the cleaned instruments or perform a secondary cleaning of the instruments to remove such deposits. The presence of corrosion, generally the formation of rust, on the instruments was also apt to occur because the water based cleaning solution had a tendency to corrode the metallic instruments being cleaned.

### SUMMARY OF THE INVENTION

In view of the shortcomings of prior tablets, there is a need for a cleaning tablet that provides not only the necessary cleaning enzymes but also the necessary concentration of those enzymes in order to sufficiently clean a given amount of soiled instruments regardless of the concentration or identity of the waste material present on those instruments. Furthermore, it is preferable that the new cleaning tablet have a formula designed to prevent the oxidation and corrosion of the instruments cleaned as well as preventing the formation of mineral deposition on those instruments.

The cleaning tablet of the present invention is generally formed of a carrier substrate of sodium bicarbonate and an acid, such as citric acid, the combination of which, in an aqueous solution, provides an effervescence that aids in dissolving the overall tablet and dispersing the active ingredients into the cleaning solution. The carrier may also include other excipient material.

In addition, the tablet includes a concentration of bioenzymatics, sometimes referred to as microbes. These bioenzymatics are living organisms suspended in a dried form while in the tablet. Introduction of the tablet to an aqueous solution activates the microbes which then once in contact with organic material (i.e. waste) present in the solution, identify the particular identity of the waste. After identifying the waste, the bioenzymatics begin to manufacture the particular enzymes necessary to breakdown the particular waste products identified. For example, when a bioenzymatic encounters a protein based waste product, such as blood, the bioenzymatic will self generate the particular proteolytic enzyme needed to break down that waste product. Additionally, other of the same bioenzymatic may identify fat or carbohydrate waste products in the solution. Those bioenzymatic will therefore self generate particular lipase and carbohydrase enzymes necessary for breaking down the identified fats and carbohydrates, respectively.

As discussed above, the cleaning tablets of the present invention are used to effect the cleaning of soiled dental instruments. The majority Dental instruments are manufactured of various metal alloys which contain some concentration of iron, and the instruments are cleaned, sterilized and reused on multiple occasions. As the cleaning solution is primarily water based, repeated submersion and drying of the cleaning solution of the dental instruments often leads to the development of corrosion, and specifically rust. To combat the formation of rust on the cleaned instruments, an anticorrosive compound, particularly a rust inhibitor, is included in the tablet.

Also as discussed above, the repeated submersion and drying of the instruments leads to the development of mineral deposits on the instruments when distilled water is not used in the ultrasonic cleaning machines. The use of distilled water is not only a hassle, but also expensive, particularly when compared to tap water. However, the tap water supplies provided in differing locals have differing mineral content. The tablet of the present invention includes an additive that prevents the formation of mineral deposits on the instruments. As an added benefit, the additive also prevents the formation of mineral deposits in the ultrasonic cleaning machine as well.

### DETAILED DESCRIPTION OF THE PREFERRED EMBODIMENTS

The preferred embodiment of the present invention pertains to an effervescent cleaning tablet having a quantity of bioenzymatic microbes for cleaning dental equipment in an ultrasonic cleaning machine. In use, the ultrasonic cleaning machine is filled with a quantity of liquid, such as water, and one or more tablets of the present invention are placed into the liquid thereby forming a cleaning solution. Soiled dental instruments are introduced to the cleaning solution. Bioenzymatics identify the nature of the wastes present on the soiled instruments and begin to breakdown the wastes thereby cleaning the instruments. While it is contemplated that various bioenzymatics could be used in the tablet of the present invention, it is preferred that the bioenzymatics are of the variety found in formula BEC-110 manufactured by Genesis Biosciences, 696 Winer Industrial Way, Lawrenceville, Ga. 30046. The formula contains a bacillus spore blend which attacks and breaks down a wide variety of organic compounds such as blood (protein), fats and carbohydrates. BEC-110 is a ten times concentration formula which allows for the presence of a high concentration of the bioenzymatics in the final tablet without unnecessary bulk.

As briefly discussed above, the mineral contents of water supplies often vary considerably. In order to prevent the buildup of mineral deposits on instruments cleaned in ultrasonic cleaning machines, it is sometimes recommended that the liquid used in the machines be distilled water. However, distilled water is more expensive than tap water, and is not always available. The preferred embodiment of the present invention includes a quantity of Ethylenediamenetetraacitic acid, also referred to as “EDTA.” The EDTA of the present invention, once diluted into the cleaning solution, reduces the effects of hard water on the instruments and the ultrasonic cleaning machine, thereby preventing the buildup of mineral deposits on the instruments and machine. By preventing the mineral deposits from forming, the present invention eliminates the need for secondary cleaning of the instruments to remove mineral deposits prior to the instruments' introduction to the autoclave.

The majority of dental instruments cleaned in ultrasonic cleaning machines are manufactured from a metallic alloy containing at least some iron. Repeated contact with aqueous cleaning solutions in the cleaning machines often leads to rust corrosion of the instruments. In order to prevent such corrosion, the preferred embodiment of the present invention includes an anti-rust agent. Preferably, the anti-rust agent is the S-69 formula available from Cortec Corporation 4119 White Bear Parkway, St. Paul, Minn. 55110. The S-69 formula provides corrosion protection for both ferrous and non-ferrous metals making it ideal for protecting the instrumentation often found in dentistry.

The balance of the tablet manufactured according to the preferred embodiment of the present invention is comprised of compounds for the formation of the effervescent, such as sodium bicarbonate and citric acid, additional excipient material, a surfactant (preferably a non-ionic detergent), color dye, scent and magnesium stearate (to aid in the release of compacted tablet from a compaction die). One preferable excipient is Di-Pac® Direct Compacting & Tableting Sugar provided by Domino®. The excipient aids in formation of the tablet without adversely affecting the other active ingredients.

There are a multitude of differing concentrations of the aforementioned products that may be brought together to form a tablet according to the present invention. It is preferable, however, that the tablet contains particular percentages by weight of those products for optimal cleaning performance. For example, the invention may contain a range of 55%-65% Di-Pac® sugar, 20%-30% sodium bicarbonate, 5%-7% citric acid, 3%-10% bioenzymatics, 0.25%-0.75% surfactant, 0.25%-0.75% EDTA, 0.25%-0.75% S-69 anti-corrosive compound, 0.25%-0.75% fragrance and between 0.10% and 0.40% each color dye and magnesium stearate. Each of the aforementioned ingredients may be found in dried form. Preparing the tablets is accomplished though blending the proper proportions of each of the ingredients and then compressing the ingredients in a tablet dye in order to form tablets. The tablets may then be released from the dye and packaged. Generally, the preferred tablet sizes have a total weight of approximately 2.5 to 3 grams.

It is additionally contemplated that the fragrance may be added as a liquid, preferably instead of adding it as a dry component. Adding the fragrance as a liquid provides a stronger scent in the finished tablet than does the use of dried fragrance. In order to maximize the fragrance in the tablet, it is contemplated that all of the dried ingredients, but not including the fragrance, are mixed together in the proper proportions and then compacted to form a tablet. An amount of a liquid fragrance, such as corn mint, is then applied to the compacted tablet. Application of the fragrance may be accomplished in any number of ways, though it is contemplated that spraying the compacted tablets with a mist of fragrance provides optimal uniform coverage.

It should be appreciated by those of skill in the art that the foregoing constitutes a description of the preferred embodiments of the present invention. All references made to the invention and/or any embodiment of the invention shall be understood to be references to preferred embodiments of the invention and shall not be construed as limiting any embodiment of the invention. Furthermore, although the present invention has been described in terms of the preferred embodiments, it is to be understood that such disclosure is not intended to be limiting nor should such references be interpreted as a disclaimer of subject matter. Various alterations and modifications will be readily apparent to those of skill in the art. Accordingly, it is intended that the appended claims be interpreted as covering all alterations and modifications as fall within the spirit and scope of the invention.

What is claimed is:

1. A tablet comprising: a concentration of an effervescent, a concentration of a surfactant and a concentration of bioenzymatics.

2. The tablet of claim 1 wherein the concentration of the bioenzymatics constitutes 3%-10% by weight of the total tablet weight.

3. The tablet of claim 1 further comprising: a concentration of EDTA.

4. The tablet of claim 1 further comprising: a concentration of EDTA wherein the concentration of the boienzymatics constitutes 3%-10% by weight of the total tablet weight and the concentration of EDTA constitutes 0.25%-0.75% by weight of the total tablet weight.

5-12. (canceled)
